(19) United States
(12) Patent Application Publication
(10) Pub. No.: US 2007/0067014 A1
(43) Pub. Date: Mar. 22, 2007

(54) Method for aortic graft installation

(75) Inventors: Danian Ke, Shanghai (CN); Lizhong Sun, Shanghai (CN); Yi Zhang, Shanghai (CN); Qiyi Luo, Shanghai (CN); Qifeng Mei, Shanghai (CN)

Correspondence Address: Crockett & Crockett;Suite 400, 24012 Calle De La Plata, Laguna Hills, CA, 92653, US

(73) Assignee: MicroPort Medical Co., Ltd.

(21) Appl. No.: 11/233,865

(22) Filed: Sep. 22, 2005

(51) Int. Cl. A61F 2/06

(52) U.S. Cl. 623/1.13

(57) ABSTRACT

An method for installing a vascular graft to replace an extensive diseased portion of the aorta in single surgical procedure.

### FIELD OF THE INVENTIONS

The inventions described below relate the field of vascular grafts.

### BACKGROUND OF THE INVENTIONS

Currently, patients who suffer from total aortic aneurism disease (including the ascending aorta, the aortic arch, descending thoracic aorta and abdominal aorta) or aortic dissection are treated by total replacement of the aorta with an aortic graft in an open chest surgery. In the typical surgery, such as the elephant trunk procedure, the patient's chest is opened by thoracotomy, the diseased portions of the aorta are removed, and an artificial vascular graft is installed in its place. Because much of the descending thoracic aorta cannot be reached in the typical open chest surgery, the typical surgical method is to insert a piece of vascular graft (the elephant trunk) into the descending thoracic aorta and leave it hanging in the aorta until the patient recovers from the open chest procedure. Then, in a follow-on surgery, the patient is opened again, and the hanging end of the elephant trunk is secured to a distal point in the descending aorta. The secondary surgery is another open procedure (a left posterolateral thoracotomy), with an opening made under the fourth rib to gain access to the descending aorta, though percutaneous surgery has been suggested, as in Ivancev, Aortic Graft Device, U.S. Pat. No. 6,773,457 (Aug. 10, 2004). The secondary surgery is usually performed one or two months after the primary surgery. In this extended period between the primary surgery in which the aortic graft is implanted, and the secondary surgery in which the hanging elephant trunk is secured within the descending aorta, the patient is at risk for a host of complications, including rupture of the descending aorta.

### SUMMARY

The methods and device described below facilitate a single stage procedure for total aortic reconstructions. A stent-graft prosthesis suitable for replacement of portion of the ascending aorta, aortic arch, and descending aorta is provided with a delivery system adapted enable installation of the stent-graft in a single stage procedure. In the single stage procedure, the aorta is severed at a proximal point (which may be anywhere along the ascending aorta or aortic arch) and at a distal point along the descending aorta. An elephant graft or other graft adapted for the particular patient is secured by end-to-end anastomosis to the ascending aorta. A stent-graft is secured within the descending aorta, and the upper end of this stent-graft is sutured to the upper edge of the severed descending aorta. The distal edge of the elephant trunk or other graft is then secured to the proximal edge of the stent graft.

### BRIEF DESCRIPTION OF THE DRAWINGS

FIG. 1 is an illustration of a diseased aorta.

FIG. 2 illustrates a step in the procedure for treating the aorta.

FIG. 3 illustrates a typical installation of the graft and stent graft.

FIG. 4 illustrates installation of the stent-graft used in the descending aorta with an aortic arch graft.

FIG. 5 illustrates installation of the stent-graft used in the descending aorta with an elephant truck aortic graft.

FIG. 6 shows details of a stent-graft adapted for use in the method illustrated in FIGS. 1 through 4.

FIG. 7 shows a stent-graft with multiple supporting stents.

FIG. 8 shows a stent-graft with multiple supporting stents.

FIG. 9 shows a stent-graft with multiple ascending branches corresponding to vessels ascending from the aorta.

### DETAILED DESCRIPTION OF THE INVENTIONS

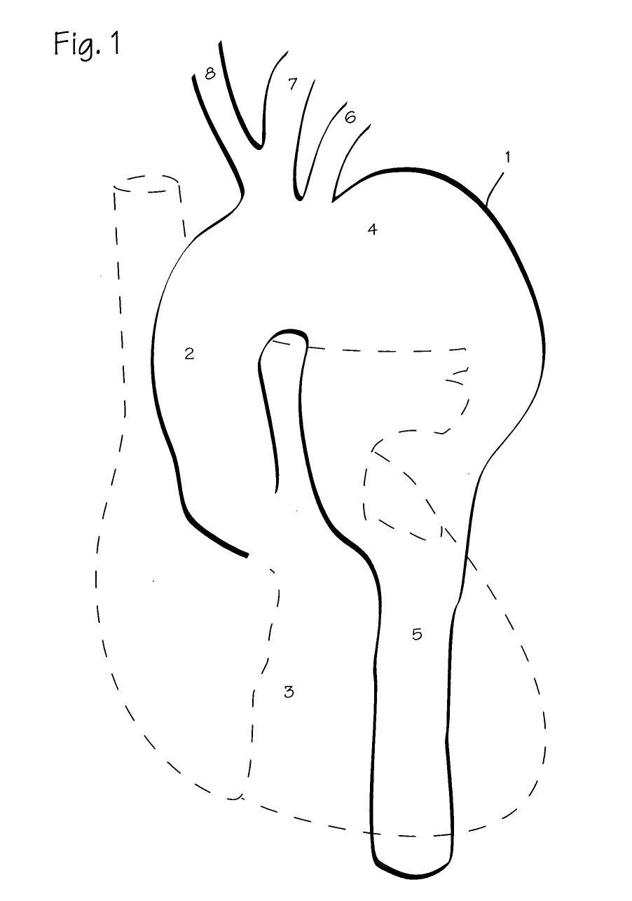

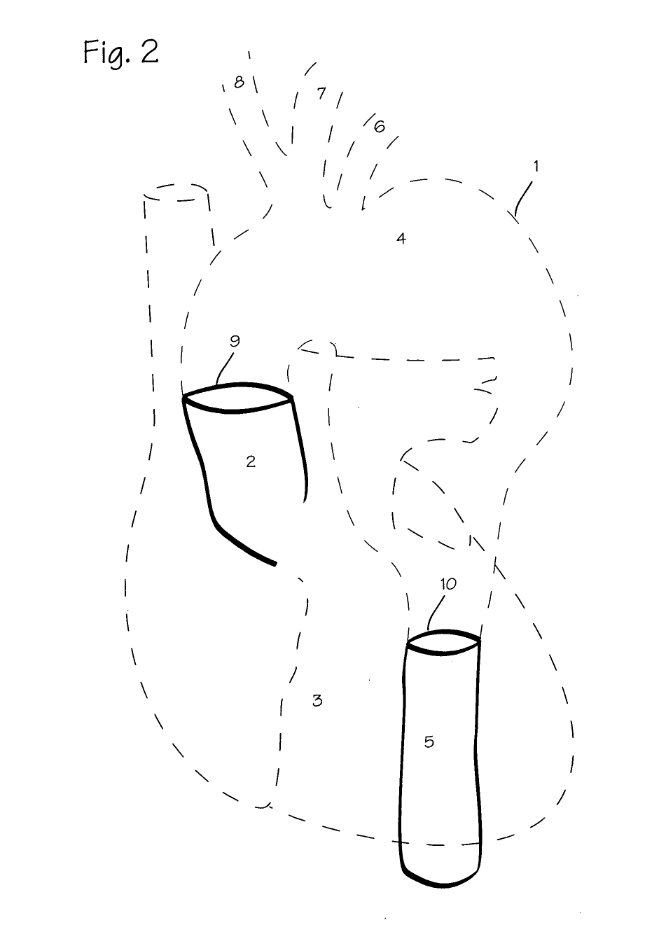

FIG. 1 illustrates a diseased aorta and its environment in the body. The aorta 1 is the main artery of the body, and includes ascending aorta 2 leading from the heart 3, the aortic arch 4, and the descending aorta 5. Several vessels branch off from the aortic arch, including the left subclavian artery 6, the left common carotid artery 7, and the brachio-cephalic trunk 8. In this illustration, the a ascending aorta, aortic arch and descending aorta have become aneurysmatic and dilated. The only treatment for this condition is replacement of the aneurysmatic portions of the aorta with a vascular graft. FIG. 2 illustrates excision of the disease portion of the aorta, at a proximal cut 9 dissecting the ascending aorta and a distal cut 10 dissecting the descending aorta.

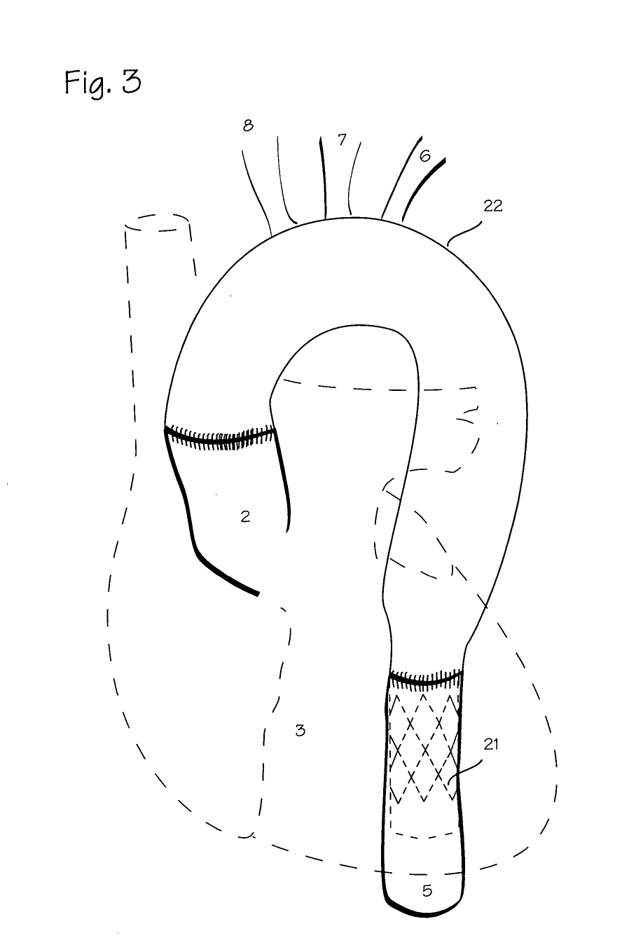

Placement of the necessary grafts and stent grafts is illustrated in FIG. 3, in which the excised portion of the aorta has been replaced by stent graft 21 and vascular graft 22. The graft extends from a proximal anastomosis to the ascending aorta at cut 9 to the distal anastomosis to the descending aorta at cut 10 and the upper (proximal) edge of the stent-graft 23. The graft and stent-graft assembly is installed in a single step procedure. In a single open chest procedure, the proximal edge of the graft may be secured to the ascending aorta, and the distal edge of the graft may be secured to the proximal edges of both the stent graft and the descending aorta. The stent graft is installed in the descending aorta by inserting it from above, through the open chest surgical space, such that the upper edge of the stent-graft and the upper edge of the descending aorta are flush or coterminous. The upper edges of both the stent-graft and dissected descending aorta are then stitched, in an end-to-end anastomosis, to the distal edge of the graft. The stent graft serves to secure the graft to the remaining descending aorta, an close any dissections or false lumens remaining in the descending aorta. Ideally, both the graft and the stent graft are installed in the same intra-operative time frame, or a single open chest procedure, to eliminate the delay, cost, and risk attendant in the typical elephant trunk procedure. After the procedure, the patient's chests is closed, without need for secondary surgery as the descending aorta is fully secured and any dissections of the descending aorta are fully addressed by the stent-graft.

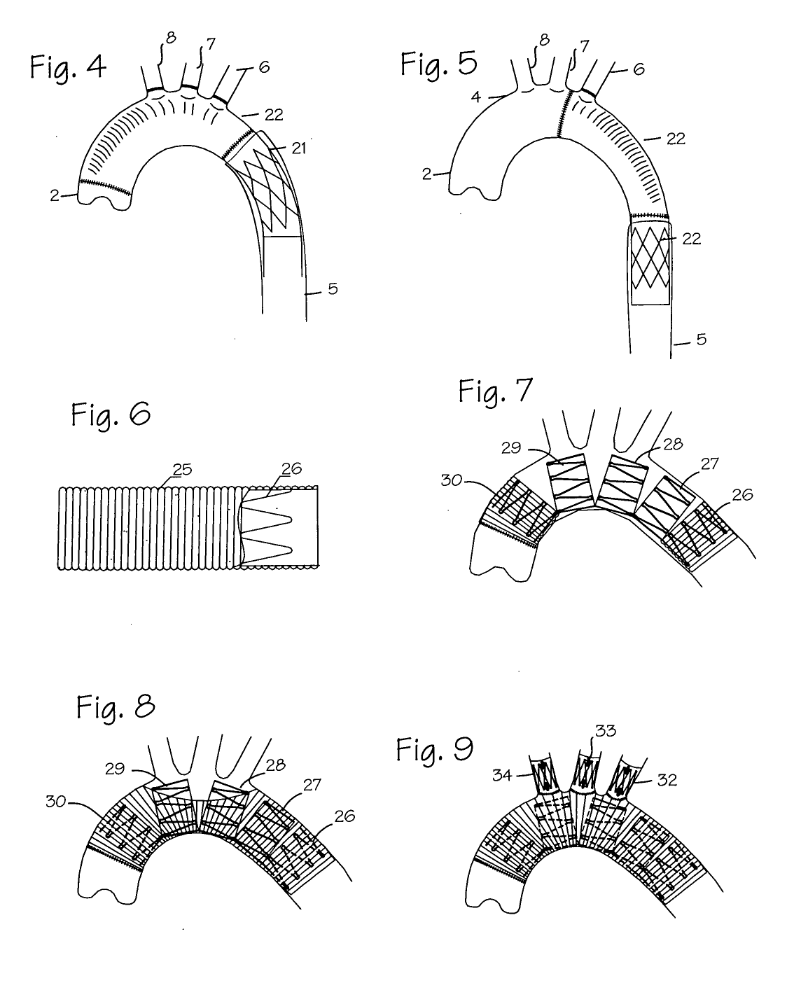

FIGS. 4 and 5 illustrate alternative configurations of grafts and stent-grafts, adapted to accommodate aortic replacement of different extent. In FIG. 4, the ascending aorta and aortic arch have been replaced with graft 22, while the descending aorta has been retained. The stent graft 21 has been installed in the upper region of the descending aorta, and secured, along with the upper margin of the dissected aorta, to the distal edge of the graft with an end-to-end anastomosis. The left subclavian artery 6, the left common carotid artery 7, and the brachi-cephalic trunk 8 are secured to suitable ports in the graft with an typical end-to-side anastomosis. In FIG. 5, the bulk of the aortic arch has been retained, and the graft is secured between the dissected terminus of the aortic arch and the upper edge of the descending aorta.

FIGS. 7, 8, 9 and 10 illustrate details of the stent-graft 24 comprised of a vascular graft 25 with supporting stents (or articulating stent segments) 26, 27, 28, 29, and 30. As shown in FIGS. 7 and 8, the graft may include open portions in the side wall of the graft, such that stents or stent segments 27, 28 and 29 which are intermediate to the terminal stents or stent segments 26 and 30 are exposed through the side wall. In FIG. 7, the entirety of the intermediate stents are exposed, while in FIG. 8 only a small circumferential portion of intermediate stents 27, 28 and 29 are exposed through side wall opening 31. FIG. 9 illustrates a stent-graft with multiple ascending side branches 32, 33 and 34 extending upwardly from the main graft. Each side branch comprises a stent-graft, with small stent segments covered with graft material.

The stent may be made of any suitable material, such as nitinol (in either pseudoelastic or shape memory form), CoCr alloy, or 316L/317L stainless steel, and may be balloon expandable or self-expanding. The stent can be of zigzag shape structure or woven structure, and may be articulated as necessary to permit the overall stent-graft to conform to the anatomy of the patient. The graft material (the material covering the stent) may any suitable vascular graft material, including ePTFE, Dacron®, PET, and various other materials and polymers. The graft surface can be smooth or corrugated.

While the preferred embodiments of the devices and methods have been described in reference to the environment in which they were developed, they are merely illustrative of the principles of the inventions. Other embodiments and configurations may be devised without departing from the spirit of the inventions and the scope of the appended claims.

We claim:

1. A method of placing a stent-graft in the aorta of a patient, said method comprising the steps of:
providing an aortic graft with a proximal end adapted for anastomosis to a distal edge of a dissected ascending aorta and a distal end adapted for anastomosis to a proximal edge of a dissected descending aorta;
providing a stent-graft sized and dimensioned for installation in the descending aorta;
performing a theracotomy on the patient to create an open surgical field in the patient's chest;
dissecting diseased portions of the aorta, thereby creating a proximal cut dissecting the ascending aorta and a distal cut dissecting the descending aorta;
installing the stent-graft having a proximal edge and a distal edge in the descending aorta by direct placement through the open surgical field, and into the descending aorta through the distal cut;
securing the proximal end of the graft to the proximal cut dissecting the ascending aorta;
securing the stent graft proximal edge to the distal end of the graft and to the descending aorta proximate the distal cut; said step of securing the stent graft being performed intra-operatively with the step of securing the proximal end of the graft to the proximal cut dissecting the ascending aorta;
closing the patient's chest.
